(19) United States
(12) Patent Application Publication
(10) Pub. No.: US 2024/0366907 A1
(43) Pub. Date: Nov. 7, 2024

(54) APPARATUS FOR DELIVERING GASEOUS NITRIC OXIDE IN PROPORTIONAL OR PULSED MODE

(71) Applicant: INOSYSTEMS, Antony (FR)

(72) Inventors: Frederic MARCHAL, Antony (FR); Thierry BOULANGER, Antony (FR); Yann BLANDIN, Bagneux (FR); Mary SCHMITT, Bagneux (FR)

(21) Appl. No.: 18/638,741

(22) Filed: Apr. 18, 2024

(30) Foreign Application Priority Data
May 4, 2023 (FR) 2304471

(51) Int. Cl. A61M 16/20; A61M 16/00; A61M 16/12

(52) U.S. Cl. CPC A61M 16/203; A61M 16/12; A61M 2016/0033; A61M 2202/0275

(57) ABSTRACT

The invention relates to an apparatus for supplying NO (1), such as an NO/N₂ gaseous mixture, comprising a main gas circuit (40; 40.1, 40.2) with flow rate control means (42, 43) comprising at least one main solenoid valve (42) operated by the operating means (50) in order to control the flow rate of NO/N₂ mixture passing through said at least one main solenoid valve (42). The main solenoid valve (42) is a proportional solenoid valve having degrees of opening of between 0 and 100%, and is configured to function in at least two given operating ranges corresponding to different degrees of opening of the main solenoid valve (42). The operating means (50) are configured to control the main solenoid valve (42) in pulsed mode in one of the operating ranges so as to deliver successive gas pulses (IG) and in proportional mode in another operating range.

### CROSS-REFERENCE TO RELATED APPLICATIONS

This patent application claims priority to French Patent Application No. 2304471, filed May 4, 2023 and the entire contents of which are incorporated herein by reference.

### TECHNICAL FIELD

The invention relates to a device or apparatus for supplying or delivering gaseous nitric oxide (NO), and an installation for administering NO-based therapeutic gas to a patient comprising such a device or apparatus for supplying NO.

### BACKGROUND

Inhaled nitric oxide, or NOi, is a gaseous medicament commonly used to treat patients suffering from acute pulmonary hypertension, in particular pulmonary vasoconstrictions in adults or children, including the newborn (PPHN), as described for example in EP-A-560928 or EP-A-1516639.

An installation for implementation of treatment by NOi, commonly known as an installation for administering NO-based therapeutic gas to a patient or more simply as an NO-administration installation, conventionally comprises at least one cylinder of NO/N₂ mixture supplying a device for supplying NO, which supplies the NO/N₂ mixture with a controlled flow rate, a medical ventilator, i.e., a respiratory assistance apparatus, for supplying a respiratory gas containing at least approximately 20 to 21% vol. of oxygen, such as an O₂/N₂ mixture or air, to which the NO in NO/N₂ form is added, circuit elements, for example at least one flexible gas duct, to convey the gaseous flows between these different items of equipment and to the patient, and a respiratory interface, such as a tracheal intubation tube, to supply the gaseous mixture containing the NO to the patient. It is also possible to provide a gas humidifier in order to humidify the gaseous mixture before it is administered to the patient. Such an NO-administration installation is shown schematically in FIG. 1.

Such an NO-administration installation is used in a hospital environment to administer the NOi treatment and thus care for patients who need to inhale NO in order to treat their pulmonary hypertension. Examples of such NO administration installations are given in documents EP-A-3821929, WO-A-2012/094008, US-A-2015/320951, US-A-2015/273175, JP-A-H11192303, WO-A-02/40914 and US-A-2003/116159.

In order to effectively deliver gas to the patient or patients, the apparatus for supplying NO, which forms part of the NO-administration installation, is generally equipped with proportional solenoid valve actuators, as taught in EP-A-659445, or all or nothing solenoid valves, or a combination of the two, as described by EP-A-375671, which allow different flow rate values to be generated depending, in particular, on the concentration of NO to be delivered, as set by the medical staff, and the concentration in the cylinders. In particular, the higher the concentration in the cylinders, the lower the flow rate of NO to be delivered.

It is therefore necessary to use actuators that are extremely precise in the low NO flow rate ranges, in particular when treating a child or newborn, but that also allow higher flow rates to be delivered for treating adults who require higher concentrations of NO.

However, this gives rise to problems and disadvantages. Indeed, in order to be able to cover the different flow rate ranges required, it is necessary to increase the number of actuators, typically by installing them in parallel, which makes the internal gas circuit of the apparatus for supplying NO more complex and inevitably more expensive.

In other words, there is a need for an apparatus or device for supplying NO, typically an NO/N₂ gaseous mixture, which does not have some or all of the aforementioned disadvantages and problems, which is suitable for the treatment of patients suffering in particular from pulmonary hypertension.

### SUMMARY

A solution according to the invention relates to an apparatus or device for supplying (i.e., delivering) NO, typically an NO/N₂ gaseous mixture, comprising:
a main gas circuit for conveying an NO/N₂ gaseous mixture,
flow rate control means comprising at least one main solenoid valve, arranged on said main gas circuit, and
operating means configured to control at least said at least one main solenoid valve in order to control the flow rate of NO/N₂ gaseous mixture passing through said at least one main solenoid valve.

Moreover, the main solenoid valve is a proportional solenoid valve having degrees of opening of between 0 and 100%, and is configured to function in at least two given operating ranges comprising at least:
a first operating range corresponding to degrees of opening of the main solenoid valve of between 0 and x % where: 0<x %≤10%, and
a second operating range corresponding to degrees of opening of the main solenoid valve greater than x % (i.e., >x %).

Moreover, the operating means are configured to control said main solenoid valve in pulsed mode (i.e., pulse modulation) in the first operating range so as to deliver successive gas pulses (IG).

According to the embodiment in question, the apparatus or device for supplying, i.e., delivering, NO of the invention may comprise one or more of the following features:
the first operating range is non-linear.
the second operating range is linear.
the operating means are configured to control said main solenoid valve in proportional mode in the second operating range, i.e., by progressively (i.e., proportionally) opening or closing the main solenoid valve in order to obtain the desired gas flow rate.
according to the embodiment in question, the first operating range corresponds to degrees of opening of the main solenoid valve of between 0 and x %, where: 0<x %≤8%, alternatively 0<x %≤5%, alternatively 0<x %≤4%, alternatively 0<x %≤3%, or alternatively 0<x %≤2%.
the operating means are configured to fix or adjust a duration or amplitude of each gas pulse delivered in pulsed mode, in order to control the flow rate of gas passing through said main solenoid valve.
the operating means are configured to adjust the pulse duration of each pulse by controlling the duration of opening of the proportional solenoid valve. When the duration is variable, the amplitude of each pulse is fixed.
the operating means are configured to adjust the pulse amplitude of each pulse by controlling the degree of opening of the proportional solenoid valve. When the amplitude is variable, the duration of each pulse is fixed.
the main, i.e., proportional, solenoid valve is traversed by an internal passage in fluidic communication with the main gas circuit.
when the main solenoid valve is at least partially open (i.e., non-zero degree of opening), the NO/N₂ gaseous mixture flows through the internal passage of the main solenoid valve either in the form of a continuous gaseous flow, or in the form of gas pulses.
the duration of opening of the proportional solenoid valve, i.e., the pulse duration, is between approximately 5 and 200 msec.
in pulsed mode, the operating means are configured to control the proportional solenoid valve to deliver successive gas pulses in which each gas pulse has a non-zero pulse duration during which the proportional solenoid valve is in a partially open position with a degree of opening x %, such that x %<10%, so as to allow a given quantity of NO/N₂ mixture to pass through during said pulse duration.
the gas pulses are separated from each other by closed time periods during which the proportional solenoid valve is in the closed position, i.e., having a degree of opening x %, such that x %=0% (i.e. closed state).
the closed time period is between 0 and 195 ms, preferably being a non-zero closed time period.
the flow rate control means can be used to control the gaseous (i.e., NO/N₂) flow in the main gas circuit.
the main gas circuit fluidically connects at least one NO inlet port to at least one NO outlet orifice in order to convey the NO/N₂ gaseous mixture from said at least one NO inlet port to said at least one NO outlet orifice.
it further comprises a bypass circuit (or line), also referred to as a “backup circuit”, which is fluidically connected to the main gas circuit upstream and downstream of said flow rate control means so as to bypass said flow rate control means,
the backup circuit comprises secondary flow rate control means for controlling the gaseous (i.e. NO/N₂) flow in the backup circuit.
the operating means are configured to control said at least one main solenoid valve of the flow rate control means and/or said at least one secondary solenoid valve of the secondary flow rate control means in order to control the flow rate of NO/N₂ mixture passing through said at least one main solenoid valve and/or said at least one secondary solenoid valve.
the secondary flow rate control means comprising at least one secondary solenoid valve, arranged on said bypass circuit.
the secondary solenoid valve is of the all or nothing (AON) type.
the AON solenoid valve is configured to adopt only two operating positions comprising an open position allowing the flow of NO/N₂ mixture to pass through and a closed position preventing any flow of NO/N₂ mixture from passing through.
it further comprises a bypass circuit (or line) which is fluidically connected to the main gas circuit upstream and downstream of the flow rate control means and comprises secondary flow rate control means comprising an all or nothing secondary solenoid valve and a fixed flow rate device.
the operating means are configured to control said at least one secondary solenoid valve, i.e., AON solenoid valve, in pulsed mode to switch said at least one secondary solenoid valve alternately to the open (i.e., maximum open) position and to the closed position so as to deliver the NO/N₂ mixture in the form of successive gas pulses.
each gas pulse comprises a pulse time period (di) of non-zero duration during which said at least one secondary solenoid valve is in the open position and allows a quantity of NO/N₂ mixture to pass through corresponding to the predetermined fixed flow rate (Q_{fixed}) of NO/N₂ mixture delivered by the fixed flow rate device during the pulse time period (di) of the gas pulse in question.
it further comprises a fixed flow rate device arranged on the bypass circuit delivering a predetermined fixed flow rate (Q_{fixed}) of NO/N₂ mixture.
the fixed flow rate device comprises a calibrated orifice.
the fixed flow rate device comprises a calibrated orifice device delivering the predetermined fixed flow rate (Q_{fixed}).
typically, the predetermined fixed flow rate (Q_{fixed}) in the backup circuit is of between 0.1 and 2 L/min, preferably between 0.1 and 1 L/min, typically of the order of 0.5 L/min.
the pulse time period (di) of each gas pulse is of non-zero variable duration, preferably of between approximately 5 msec and 200 msec.
the gas pulses are separated from each other by closed time periods (df) during which the proportional main solenoid valve and/or the secondary solenoid valve are in the closed position.
the closed time periods (df) have equal or variable durations.
typically, the closed time periods (df) have durations ranging from greater than or equal to 0 msec up to 195 msec.
it comprises storage means configured to store the predetermined fixed flow rate (Q_{fixed}) delivered by the fixed flow rate device.
the storage means are preferably integrated into the operating means.
the storage means comprise a computer memory, for example a flash memory, RAM or the like.
the fixed flow rate device is arranged on said bypass circuit, downstream of said at least one secondary solenoid valve.
the operating means are configured to determine the desired quantity of NO/N₂ mixture based on the flow rate of gas provided by a medical ventilator, the desired concentration and the NO content of the NO/N₂ mixture.
the operating means comprise at least one (micro) processor.
said at least one (micro) processor is arranged on an electronic board.
said at least one (micro) processor implements at least one algorithm.
the main gas circuit comprises said at least one main solenoid valve and at least one flow rate sensor.
said at least one flow rate sensor is arranged upstream of said at least one main solenoid valve.
said at least one flow rate sensor is electrically connected to the operating means.
said at least one main solenoid valve is a proportional solenoid valve controlled by the operating means.
it comprises a mass flow controller (MFC) comprising the main solenoid valve and the flow rate sensor arranged on the main gas circuit.
the main gas circuit comprises a main NO line.
said at least one main solenoid valve and said at least one flow rate sensor are arranged on the main NO line.
the upstream part of the main NO line branches into two parallel sections each comprising a gas inlet orifice for receiving an NO/N₂ mixture.
the bypass line is fluidically connected to the main NO line, upstream and downstream of said flow rate control means, so as to bypass said flow rate control means.
the bypass circuit further comprises a gas pressure-reducing device arranged upstream of said at least one secondary solenoid valve.

The invention also relates to an installation for supplying gas, i.e., a gas containing NO and oxygen, to a patient, comprising:
at least one source of NO/N₂ mixture, typically at least one pressurized gas cylinder containing the NO/N₂ mixture,
an apparatus for supplying NO according to the invention fluidically connected to said at least one source of NO/N₂ mixture,
a medical ventilator configured to supply a respiratory gas containing oxygen, typically at least approximately 20% oxygen, such as air or an N₂/O₂ mixture, for example at least approximately 21% oxygen,
a patient circuit to which the apparatus for supplying NO according to the invention and the medical ventilator for supplying said patient circuit with said NO/N₂ gaseous mixture and said respiratory gas containing oxygen are fluidically connected,
and a flow rate sensor arranged on the patient circuit and configured to determine (i.e., measure) and supply to the operating means of the NO delivery device at least one measurement signal representative of the gas flow rate within the patient circuit, i.e., at least one flow rate or pressure signal or measurement.

According to the embodiment in question, the gas supply installation of the invention may comprise one or more of the following features:
the patient circuit comprises an NO injection module supplied with NO/N₂ mixture by the apparatus for supplying NO, preferably via an NO injection line or duct, such as a flexible gas duct.
the flow rate sensor is arranged on an inhalation branch of the patient circuit and is electrically connected to the operating means via at least one electrical connection (e.g., cables or the like).
the flow rate sensor (i.e., used to measure the flow rate of the respiratory gas supplied by the ventilator) is arranged on the patient circuit between the medical ventilator and the NO injection module.
according to one embodiment, the flow rate sensor is a mass flow sensor or the like. However, another type of sensor may be used provided that it allows a direct or indirect measurement of the flow rate to be taken.
the flow rate sensor is electrically connected to the operating means via at least one electrical connection such as cables or the like.
the apparatus for supplying NO is supplied with NO/N₂ gaseous mixture formed from NO and nitrogen from the at least one source of NO/N₂ mixture.
the NO supply line of the apparatus for supplying NO conveys the NO/N₂ gaseous mixture.
the NO/N₂ gaseous mixture from the at least one source of NO/N₂ mixture contains between 100 and 2000 ppmv of NO, typically less than 1000 ppmv of NO, the remainder being nitrogen (and possibly unavoidable impurities).
the apparatus for supplying NO comprises a housing in which the main circuit, the bypass circuit, the operating means, the main and secondary solenoid valves and other components are arranged.
power supply means supplying electric current to the components that need electrical energy in order to function, in particular the apparatus for supplying NO and the medical ventilator, such as means for connection to the mains (110/220V) and/or at least one battery or the like.
the medical ventilator comprises a motorized blower (i.e., also referred to as a turbine, compressor or the like) delivering the respiratory gas, typically air or an oxygen/nitrogen mixture, or indeed pure oxygen.
the at least one source of NO contains NO/N₂ gaseous mixture containing between 100 and 2000 ppmv of NO, the remainder being nitrogen (N₂), stored at a pressure of between 10 and 250 bar absolute, typically at more than 100 bar absolute (before the start of withdrawal), preferably between 100 and 1000 ppmv of NO, the remainder being nitrogen (N₂).
the at least one source of NO comprises at least one pressurized gas cylinder.
the source of NO comprises at least one gas cylinder having a capacity of between 0.5 and 50 L (water equivalent).
the gas cylinder comprises a cylindrical body made from steel or aluminium alloy and is equipped with a simple valve (without regulator) or a valve with an integrated regulator or IRV, preferably an IRV protected by a protective cap, for example made from metal or polymer.
the patient circuit comprises an inhalation branch and an exhalation branch;
the patient circuit comprises flexible ducts forming the inhalation branch and/or the exhalation branch, typically polymer hoses.
the inhalation branch and the exhalation branch, e.g., flexible ducts, are connected to a joining piece such as a Y-piece.
the inhalation branch and/or the exhalation branch are fluidically connected to a patient respiratory interface, preferably via the joining piece.
the patient respiratory interface comprises a tracheal intubation tube or a breathing mask.
the inhalation branch and the exhalation branch comprise flexible ducts, for example made from a polymer.
the inhalation branch and the exhalation branch are further fluidically connected respectively to inlet and outlet ports of the medical ventilator.
the inhalation branch of the patient circuit may comprise a gas humidifier arranged downstream of the NO injection module so as to be able to humidify the gas before it is administered by inhalation to the patient.
the concentration or dosage of NO in the final gaseous mixture, i.e., the gas inhaled by the patient, after the NO/N₂ mixture has been mixed with the respiratory gas containing oxygen coming from the ventilator, such as air or an O₂/N₂ mixture (approximately >20% vol.), is between 1 and 80 ppm by volume (ppmv), depending on the treated population, i.e., newborns or adults, the condition of the patient and/or the disease to be treated.

The apparatus for supplying NO and/or the installation for supplying gas of the invention are particularly well suited to use in a therapeutic treatment method implementing the administration by inhalation, in particular via a tracheal intubation tube, of a gaseous mixture comprising between 1 and 80 ppmv of NO and at least approximately 20% vol. of oxygen, typically of the order of 10 to 20 ppmv of NO, to one or more patients (e.g., adults, children, adolescents or newborns) in need of it, typically patients (i.e., human beings) suffering from pulmonary hypertension and/or hypoxia that may cause pulmonary vasoconstriction or the like, for example caused by pulmonary diseases or disorders such as PPHN (persistent pulmonary hypertension of the newborn) or ARDS (acute respiratory distress syndrome), or those caused by heart surgery in which the patient is put on extracorporeal circulation (ECC).

In the context of the invention:
“ppmv” means parts per million by volume.
“% vol.” means percentage by volume.
“NO” denotes nitric oxide.
“N₂” denotes nitrogen.
“O₂” denotes oxygen.
“Flow rate sensor” is given to mean a sensor of the type that measures and supplies at least one flow rate signal or measurement per se, or of the type that measures and supplies at least one pressure signal or measurement that is then converted into a flow rate by the operating means.
The term “means” is in all cases considered to be wholly equivalent to and capable of being substituted by the term “device”; for example, the term “operating means” can be replaced by “operating device”, the term “measurement means” can be replaced by “measurement device” and the term “control means” can be replaced by “control device”, etc.

### BRIEF DESCRIPTION OF THE VIEWS OF THE DRAWINGS

The invention will now be better understood from the following detailed description, given by way of illustration but without limitation, with reference to the appended figures, in which:

FIG. 1 schematically depicts an installation for administering NO-based therapeutic gas to a patient, incorporating a device for supplying NO according to the invention,

FIG. 2 schematically depicts the operation of the device for supplying NO according to the invention,

FIG. 3 schematically depicts the successive gas pulses resulting from pulsed mode control of the secondary solenoid valve of the device for supplying NO of FIG. 2,

FIG. 4 schematically depicts the successive gas pulses resulting from control of the width of each gas pulse in pulsed mode, during operation of the main solenoid valve in its non-linear operating range, and

FIG. 5 schematically depicts the successive gas pulses resulting from control of the amplitude of each gas pulse in pulsed mode, during operation of the main solenoid valve in its non-linear operating range.

### DETAILED DESCRIPTION

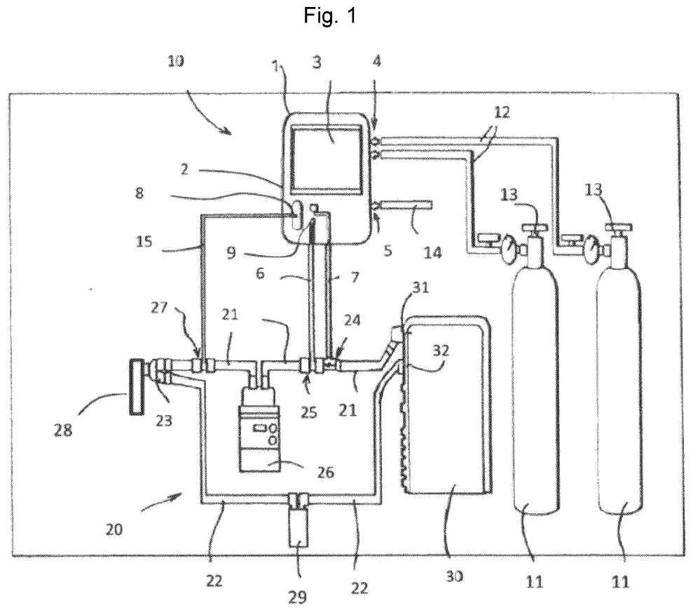

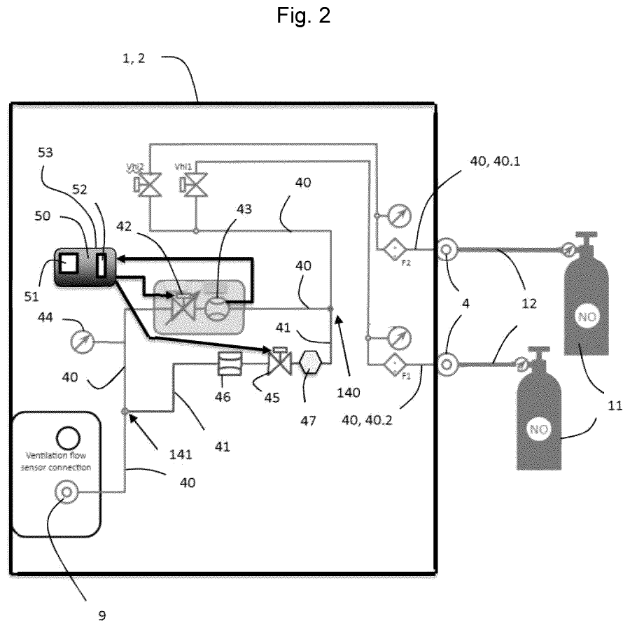

FIG. 1 schematically depicts an embodiment of an installation 10 for administering therapeutic gas, i.e., an NO-based gaseous mixture, to a patient, comprising a device for supplying NO 1 according to the present invention, in particular that schematically depicted in FIG. 2.

The installation 10 in this case comprises two gas sources that are two pressurized gas containers 11, i.e., compressed gas cylinders, arranged in parallel, each containing a gaseous mixture of NO and nitrogen (N₂), i.e., an NO/N₂ mixture, typically containing from 225 to 2000 ppmv of NO, the remainder being nitrogen (N₂), preferably 450 to 1000 ppmv of NO, stored at a pressure that may reach 150 bar, or indeed 180 or more (full container), for example an NO/N₂ mixture containing 450 ppmv or 800 ppmv of NO.

The containers 11 of NO supply the NO/N₂ mixture to a device or apparatus for supplying NO 1 according to the invention, part of the internal architecture of which is schematically depicted in FIG. 2. The connection is provided by NO feed lines 12, i.e., gas piping, such as flexible hoses or the like. Each line 12 is connected to an NO inlet port 4 of the device for supplying NO 1 in order to supply the main gas circuit 40, 40.1, 40.2 inside the housing 2 of the device for supplying NO 1 (see FIG. 2).

The device for supplying NO 1 also comprises an oxygen inlet port 5 fluidically connected, via an oxygen feed line 14, such as a flexible hose or the like, to an oxygen source (not shown), for example a pressurized oxygen container, typically a cylinder of O₂ or, alternatively, the hospital network, i.e., an oxygen feed pipe arranged in the hospital building where the patient is being treated.

The cylinders 11 of NO and the O₂ source, e.g., an oxygen cylinder, are preferably equipped with a gas distribution valve 13, preferably incorporating gas pressure regulation means, i.e., an IRV or valve with an integrated regulator, or other systems for controlling and/or regulating gas so as to be able to control the flow rate and/or the pressure of the gas that they deliver, for example in order to supply an NO/N₂ mixture and/or oxygen at a pressure of between 3 and 6 bar. The gas distribution valve 13 may be protected against impacts by a protective cap (not shown).

Moreover, the installation 10 also comprises a medical ventilator 30, i.e., a respiratory assistance apparatus, supplying a flow of respiratory gas containing oxygen in a non-hypoxic quantity to the patient P, i.e., at least approximately 21% vol. of oxygen, typically air, oxygen or an oxygen/nitrogen mixture (N₂/O₂).

The medical ventilator 30 is fluidically connected to the patient via a respiratory gas circuit 20 which in this case has two respiratory branches 21, 22, since it comprises an inhalation branch 21, i.e., a gas supply line, which is used to convey the respiratory gas to the patient, and an exhalation branch 22, which is used to recover the CO₂-enriched gas exhaled by the patient.

The inhalation and exhalation branches 21, 22 typically comprise flexible hoses made from polymer or the like. The inhalation branch 21 is fluidically connected, upstream, to a gas outlet 31 of the medical ventilator 30 and, downstream, to a joining piece 23, typically a Y-piece. Similarly, the exhalation branch 22 is fluidically connected, upstream, to the joining piece 23 and, downstream, to a gas inlet 32 of the medical ventilator 30. The inhalation and exhalation branches 21, 22 are therefore connected to each other at the joining piece 23, typically a Y-piece, which is itself in fluidic communication with a respiratory interface 28 supplying the gas to the patient, such as a tracheal intubation tube, a breathing mask or the like, preferably a tracheal intubation tube.

It will be appreciated that the medical ventilator 30 and the device or apparatus for supplying NO 1 are normally supplied with electricity by at least one source of electric current, in particular their components which require electrical energy in order to function, in particular the operating means 50 and the screen 3 of the device for supplying NO 1, the control system (not shown) of the medical ventilator 30, i.e., an electronic board with at least one microprocessor, or any other component, in particular the motorized internal turbine that supplies the flow of air or the like, i.e., the respiratory gas. The source of electric current can be the mains (110/220V) and/or an electric battery, which is preferably rechargeable.

As can be seen, the device or apparatus for supplying NO 1 makes it possible to inject the NO/N₂ mixture into the inhalation branch 21 of the gas circuit 20, via an NO injection line or duct 6 opening into the inhalation branch 21 at an injection site 25, so as to mix (i.e., dilute) at that location the flow of NO/N₂ and the flow of respiratory gas containing at least approximately 21% O₂, i.e. air or oxygen/nitrogen mixture, delivered by the medical ventilator 30.

The device for supplying NO 1 comprises an outlet orifice or port 9 situated at the outlet of its main gas circuit 40, 40.1, 40.2 through which the flow of NO/N₂ exits the housing 2 of the device for supplying NO 1 and enters the NO injection duct 6 which is fluidically connected to said outlet orifice or port 9, for example via a connector or the like.

The therapeutic gaseous mixture obtained (i.e., at the injection site 25 and downstream of the inhalation branch 21), i.e., the final mixture, therefore contains oxygen (approximately >20 to 21% vol.), generally nitrogen, and a variable and adjustable concentration of NO, typically between 1 and 80 ppmv, as a result of the dilution that occurs upon mixing the gaseous flows, and possibly unavoidable impurities, such as argon, for example, which may be comprised in the gas but which are not desirable, in particular when the flow of gas from the ventilator 30 is atmospheric air rather than an O₂/N₂ mixture.

Advantageously, a gas humidifier 26 is also provided, arranged on the inhalation branch 21 downstream of the injection site 25, in order to humidify the flow of therapeutic gas, e.g., the final NO/N₂/O₂ mixture, by the addition of water vapour, before it is inhaled by the patient, which makes it possible to avoid or limit drying of the patient's airways during his or her treatment by inhalation of the gas. According to another embodiment, the gas humidifier 26 could also be arranged upstream of the injection site 25.

Moreover, optionally, the exhalation branch 22 used to recover the CO₂-enriched exhaled gases may comprise one or more other optional components, such as, for example, a device 29 for eliminating CO₂ and/or water vapour, i.e., a CO₂ and/or water trap, such as a hot container, a filter or the like, which is used to remove (at least some of) the CO₂ and/or water vapour present in the gas exhaled by the patient.

As shown in FIG. 1, on the inhalation branch 21, upstream of the injection site 25, a flow rate sensor 24 is also provided, for example a mass flow sensor, connected to the device for supplying NO 1, in particular to the operating means 50 of said device for supplying NO 1, via a respiratory gas flow rate measurement line 7 used to measure or determine the flow rate of gas from the ventilator 30 within the inhalation branch 21 and supplying it to the operating means 50 in order for the latter to be able to control the quantity de NO/N₂ to be supplied.

Indeed, determining the flow rate of gas from the ventilator 30 makes it possible, in particular, to regulate the passage of NO through the device for supplying NO 1, i.e., to be able to choose the flow rate of NO/N₂ mixture to be injected according to the desired NO content, the composition of the NO/N₂ mixture from the cylinders and the flow rate of gas (i.e., air or air/O₂) from the ventilator 30. The way in which the passage of NO through the device for supplying NO 1 is regulated is detailed hereinafter in reference to FIG. 2 in particular.

Moreover, a gas sampling line 15 fluidically connects the device for supplying NO 1, via a sampling inlet 8, to the inhalation branch 21 of the respiratory gas circuit 20, via a connection component 27, preferably in the vicinity of the Y-piece 23, for example approximately 10 to 20 cm upstream of the Y-piece. This sampling line 15 is used to take gas samples from within the inhalation branch 21 in order to check whether they comply with the desired gaseous mixture that needs to be administered to the patient, in particular in terms of its NO content but also the amount of O₂ that it contains, and indeed toxic NO₂ species that may have been created by oxidation of NO molecules by oxygen.

As mentioned above, the NO/N₂ mixture delivered by the device for supplying NO 1 is injected (at 25) into the respiratory flow containing at least 21% vol. of oxygen (e.g. air or O₂/N₂ mixture) from the medical ventilator 30 before being administered to the patient by inhalation in the form of a final respiratory mixture (i.e., NO/N₂/O₂ or NO/N₂/air mixture) generally containing a few ppmv or a few dozen ppmv of NO (ppm by volume) and at least approximately 21% vol. of oxygen O₂, for example of the order of 1 to 80 ppmv of NO, the remainder essentially generally being nitrogen (N₂), typically less than 40 ppmv of NO, for example of the order of 20 ppmv.

In order to deliver the correct quantity of NO, it is necessary to be able to adjust the quantity, i.e., the flow, of NO/N₂ mixture passing through the device for supplying NO 1 and exiting through the NO outlet orifice 9 before being conveyed through the NO injection line 6 to the injection site 25. This adjustment is carried out by the operating means 50 based not only on the measurements of the flow rate of gas (i.e., air or O₂/N₂ mixture) from the ventilator 30, which are taken by the flow rate sensor 24 arranged on the inhalation branch 21, but also on the desired amount or dose of NO set by the medical staff and the composition of the NO/N₂ mixture from the cylinders 11 of NO.

To this end, as shown in FIG. 2, the device or apparatus for supplying NO 1 comprises a main internal gas circuit 40; 40.1, 40.2, i.e. passages, ducts or the like, for conveying the NO/N₂ mixture entering through the at least one gas inlet port or orifice 4 to the at least one NO outlet orifice 9, to which the NO injection duct 6 is fluidically connected.

In the proposed embodiment, the main gas circuit 40; 40.1, 40.2 comprises a main NO line 40 whose upstream part branches into two parallel sections 40.1, 40.2 each comprising a gas inlet orifice 4 fluidically connected to one or other of the cylinders 11 of NO/N₂ mixture that supplies it with the NO/N₂ mixture at the desired pressure, for example between 3 and 6 bar.

The main gas circuit 40; 40.1, 40.2, in particular the main NO line 40, comprises NO/N₂ flow rate control means 42, 43 which are operated by the operating means 50 provided with a (micro) processor 52 of the device for supplying NO 1. The operating means or device 50 typically comprise at least one (micro) processor 52 arranged on at least one electronic board 53, the operation of which is explained hereinafter.

Moreover, a bypass circuit 41, also referred to as a “backup circuit”, is fluidically connected (at 140, 141) to the main gas circuit 40; 40.1, 40.2, in particular to the main NO line 40, at an upstream connection site 140 situated upstream of said flow rate control means 42, 43 and at a downstream connection site 141 situated downstream of said flow rate control means 42, 43, as seen in the normal direction of flow of the NO/N₂ mixture (from the inlets 4 towards the outlet 9), in such a way as to bypass said flow rate control means 42, 43.

In other words, the bypass circuit 41 comprises or constitutes a bypass line that can be used to divert the NO/N₂ mixture and prevent it from passing through the flow rate control means 42, 43 arranged on the main gas circuit 40; 40.1, 40.2, in particular on the main NO line 40. This bypass or backup circuit 41 is used in the event of a malfunction, in particular of the main solenoid valve 42 arranged on the main gas circuit 40; 40.1, 40.2, in order to ensure NO/N₂ mixture is delivered despite this malfunction.

The bypass circuit 41, i.e. gas passage, gas duct or the like, comprises secondary flow rate control means 45, 46, i.e. a secondary flow rate control device, comprising at least one secondary solenoid valve 45, and a fixed flow rate device 46, i.e., a device delivering or supplying (downstream) a predetermined fixed flow rate of gas (Q_{fixed}). Optionally, the flow rate could be modified, in particular by using a pressure-reducing device 47, as explained hereinafter.

According to the invention, the secondary solenoid valve 45 is an all or nothing (AON) solenoid valve, i.e., it is configured to adopt only two operating positions (i.e., open/closed), i.e. an “open position” allowing the entire flow of NO/N₂ mixture to pass through and a “closed position” preventing any flow of NO/N₂ mixture from passing through. For example, the AON solenoid valve available under the commercial reference FAS 10 mm PICOSOL may be used as the AON solenoid valve 45.

In other words, when the all or nothing (AON) solenoid valve 45, i.e., the secondary solenoid valve 45, is in an open position referred to as the “open position”, the entire gaseous flow of NO/N₂ mixture can pass through it and flow freely through the bypass circuit 41 in the normal direction of flow of the gas, i.e., in the direction extending from the upstream connection site 140 towards the downstream connection site 141.

Conversely, when the AON solenoid valve, i.e., the secondary solenoid valve 45, is in a closed position referred to as the “closed position”, the gaseous flow of NO/N₂ mixture is interrupted and can no longer pass through it because it is stopped by this solenoid valve 45. The free flow of the gas in the bypass circuit 41 is prevented, stopped or impossible.

The secondary solenoid valve 45 is controlled by the operating means 50 provided with a processor 52 so as to switch it from one position to the other, preferably to deliver NO in pulses or bursts, as detailed below.

Moreover, a fixed flow rate device 46, typically a calibrated orifice device, is arranged on said bypass circuit 41. It is used to deliver a predetermined fixed flow rate Q_{fixed} of NO/N₂ gaseous mixture, i.e., a known flow rate, for example a flow rate of 0.1 to 2 L/min, for example of the order of 0.5 L/min. It is arranged downstream of the secondary solenoid valve 45, i.e., between the secondary solenoid valve 45 and the downstream connection site 141. The fixed gas flow rate Q_{fixed} resulting from the gaseous flow passing through the fixed flow rate device 46 is known and stored in storage means 51 configured to store the fixed flow rate Q_{fixed}. The storage means 51 are preferably integrated into the operating means 50, in particular carried by the electronic board 53. The storage means 51 comprise at least one computer memory, such as a flash memory or the like.

The bypass circuit 41 may preferably also comprise a gas pressure-reducing device 47 which is arranged upstream of the secondary solenoid valve 45 and is used to regulate or adjust the pressure of the gas, typically reducing it, if necessary, and/or the flow rate of gas.

In all cases, the storage means 51 cooperate with the operating means 50, in particular with the at least one processor 52, in particular to provide it with the stored fixed gas flow rate value Q_{fixed}. The storage means 51 use the stored fixed gas flow rate value Q_{fixed} (i.e., at least one value) in order to perform calculations, in particular of the pulse time period (di), as explained hereinafter, when it is appropriate to vary the quantity of NO supplied.

Furthermore, the flow rate control means 42, 43 comprise a main proportional solenoid valve 42 and a flow rate sensor 43, i.e., means or a device for measuring flow rate, arranged on the main gas circuit 40; 40.1, 40.2, typically on the main NO line 40 of said main gas circuit 40; 40.1, 40.2. For example, the proportional solenoid valve available under the commercial reference FAS 16 mm FLATPROP may be used as the proportional solenoid valve 42.

The flow rate sensor 43 is preferably arranged upstream of the proportional solenoid valve 42, as seen in the direction of the gas flow from the at least one inlet orifice 4 towards the outlet orifice 9, i.e., from the upstream connection site 140 towards the proportional solenoid valve 42. The flow rate sensor 43 and the proportional solenoid valve 42 are electrically connected to the operating means 50.

The flow rate sensor 43 provides the operating means 50 with measurements representative of the gas flow rate in the main NO line 40 of the main gas circuit 40; 40.1, 40.2 immediately upstream of the proportional solenoid valve 42. Their precise operation is explained below.

Generally, the operating means 50 are configured to control the main solenoid valve 42 and the secondary solenoid valve 45 in order to control the flow rate of NO/N₂ mixture passing through this main solenoid valve 42 and secondary solenoid valve 45, in particular in order to allow the gaseous flow to flow through one or the other of the main solenoid valve 42 (during normal operation) and secondary solenoid valve 45 (during backup operation, i.e., in the event of a malfunction) but never through both at the same time. In other words, the main solenoid valve 42 and the secondary solenoid valve 45 are never open simultaneously, meaning that the gaseous flow must necessarily flow exclusively through one of the two.

The components of the apparatus 1 are arranged in a housing 2, i.e., a rigid external carcass.

Normally, i.e., during normal operation of the NO delivery apparatus 1, the gaseous flow of NO/N₂ gaseous mixture flows through the main gas circuit 40, 40.1, 40.2, in particular through the main NO line 40, and passes through the main solenoid valve 42 because this main solenoid valve 42 is open in order to allow the gas to flow through the main gas circuit 40, 40.1, 40.2, in particular through the main NO line 40, while the secondary solenoid valve 45 is closed in order to prevent any gas from flowing through the bypass circuit 41 which comprises the secondary solenoid valve 45.

Conversely, in the event of a problem, for example if there is a fault in the flow rate sensor 43 or the main solenoid valve 42 malfunctions, the gaseous flow of NO/N₂ gaseous mixture is diverted towards the bypass circuit 41, which then acts as a backup circuit. The main solenoid valve 42 then switches to the closed position to stop/prevent the gaseous flow from flowing through the main solenoid valve 42, i.e., through the portion of the main NO line 40 comprising the flow rate control means 42, 43, including the main solenoid valve 42, which is located schematically between the upstream connection site 140 and the downstream connection site 141.

In order to be able to better control the flow rate of the NO/N₂ gaseous mixture supplied by the NO delivery apparatus 1, i.e., delivered in particular through the outlet orifice or port 9 that is situated at the outlet of its main gas circuit 40, 40.1, 40.2, when this gaseous flow flows through the bypass circuit 41, i.e., in backup mode, the secondary solenoid valve 45, which is an all or nothing (AON) solenoid valve, is controlled in pulsed mode by the operating means 50, i.e., it does not deliver a continuous flow of NO/N₂ but small doses or pulses (i.e., bursts) of gaseous NO/N₂.

When the operating means 50 operate the secondary solenoid valve 45 to deliver gas pulses or “bursts” of gas into the bypass circuit 41, i.e., downstream of the secondary solenoid valve 45, the main solenoid valve 42 is closed to prevent the gaseous flow, i.e., the flow of NO/N₂ mixture, from passing through it.

More precisely, the operating means 50 are configured, e.g. programmed, to control the secondary solenoid valve 45 in pulsed mode in such a way as to cause it to switch alternately, i.e., over time, to the open position and to the closed position and to thus deliver the NO/N₂ mixture in the form of successive gas pulses, i.e., “bursts” of gas.

The gas pulses can be controlled. Indeed, each gas pulse comprises or is characterized by a pulse time period (di) of non-zero duration (i.e., >0 msec) during which the secondary solenoid valve 45 is in the open position and thus allows a desired quantity of NO/N₂ mixture to pass through, i.e., it allows a given quantity of gas to pass through it, towards the fixed flow rate device 46 arranged on said bypass circuit 41, downstream of the secondary solenoid valve 45, which is an AON solenoid valve controlled by the operating means 50.

The desired quantity of NO/N₂ mixture is therefore variable because it corresponds to the predetermined fixed flow rate (Q_{fixed}) of NO/N₂ mixture delivered by the fixed flow rate device 46 during the pulse time period (di) of the gas pulse in question, i.e., the time during which the secondary solenoid valve 45 is in the open position and allows gas to pass through.

In other words, the proportion or quantity of NO/N₂ mixture passing through the AON secondary solenoid valve 45 may be controlled by adjusting the pulse time period (di) of each gas pulse in question because the fixed flow rate device 46 delivers a predetermined, i.e., known, fixed flow rate (Q_{fixed}) of NO/N₂ mixture.

The pulse time period (di) of each gas pulse is calculated or determined by the operating means 50 depending, in particular, on the desired gas flow rate (Q_{desired}) and also on the NO content of the NO/N₂ mixture and at least one predefined and generally stored flow rate, in particular in the event of failure of the sensor 24.

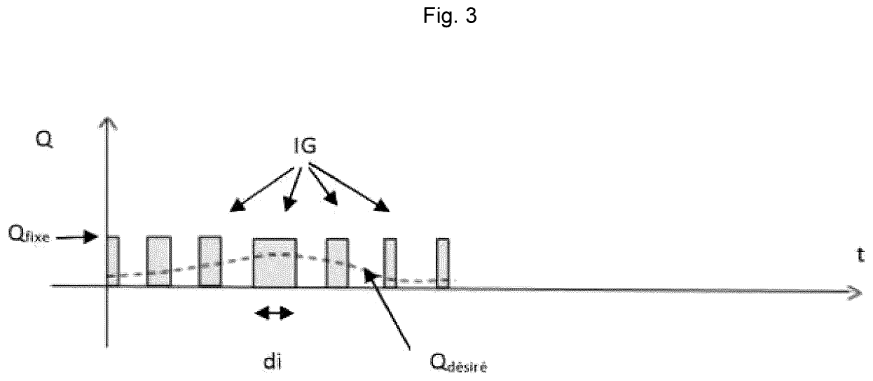

Therefore, FIG. 3 schematically depicts a plurality of successive gas pulses IG produced over time (t) by controlling the opening/closing of the secondary AON solenoid valve 45 in pulsed mode.

It can be seen that each gas pulse IG has a pulse duration or time period (di) that is non-zero (i.e., >0 msec) but that can be varied so as to deliver variable doses or quantities of NO/N₂ mixture and thus obtain a desired gas flow rate (Q_{desired}) downstream of the fixed flow rate device 46. The pulse time period (di) can typically reach approximately 200 msec.

Moreover, according to the invention, during normal operation (i.e., when there are no malfunctions), when this gaseous flow is not flowing through the bypass circuit 41, the flow passing through the proportional solenoid valve 42 arranged on the main gas circuit 40, 40.1, 40.2 is controlled proportionally.

The proportional solenoid valve 42 is generally configured to have degrees of opening ranging from 0 to 100% where:
0% corresponds to the solenoid valve 42 being totally closed, preventing any gaseous flow from passing through,
100% corresponds to the solenoid valve 42 being totally open, allowing a maximum flow rate of the gaseous flow, and
the intermediate values between 0 and 100% correspond to intermediate degrees of opening, i.e., the solenoid valve 42 being partially open, allowing non-zero gas flow rates lower than the maximum flow rate of the gaseous flow to pass through.

In this case, the operating means 50 are configured to control the proportional solenoid valve 42 differently depending on whether this proportional solenoid valve 42 is in its linear operating range or in its non-linear operating range.

Indeed, the proportional solenoid valve 42 is configured to operate in (at least) two given operating ranges between 0 and 100%, i.e.:
a first, i.e., non-linear, operating range, also referred to as the “low” operating range corresponding to degrees of opening of the proportional solenoid valve 42 of between 0 and x % where: 0<x %≤10%, preferably 0<x %≤5%, for example between 0 and 1% (i.e., x %=1%), and
a second, i.e., linear, operating range, corresponding to degrees of opening greater than x %, i.e., between x % and at least 85%, for example between x % and 90%, for example between 1 and 90%, typically between 10 and 90% of its operating range.

The proportional solenoid valve 42 may optionally be configured to operate in a third, i.e., non-linear, operating range, also referred to as the “high” operating range corresponding to degrees of opening close to 100%, for example at least 80%, preferably at least 90%, for example between 90% and 100%.

In all cases, according to the invention, the operating means 50 are configured to control the proportional solenoid valve 42 to deliver a supply of gas, i.e., NO/N₂ mixture, in a different manner depending on the operating range in question, i.e., depending on the degree of opening of the proportional solenoid valve 42, i.e.:
in proportional mode in the second operating range, i.e., the linear range, i.e., for degrees of opening greater than x %, typically between 1 and 90%, for example between 1% and 90% (x %=1%) or between 10% and 90% (x %=10%), and
in pulsed mode or in pulse modulation in the first operating range, i.e., the non-linear range, i.e., between 0 and x %, where x %≤10%, for example between 0 and 10% (x %=10%) or between 0 and 1% (x %=1%).

Naturally, x % may represent values between 1 and 10%, for example 2%, 3%, 4%, 5%, 7% etc. or the like.

Therefore, it seems that:
in proportional mode, the gaseous flow passing through the solenoid valve 42 is continuous
in pulsed mode, the gaseous flow passing through the solenoid valve 42 is in the form of successive gas pulses (IG), and is therefore discontinuous.

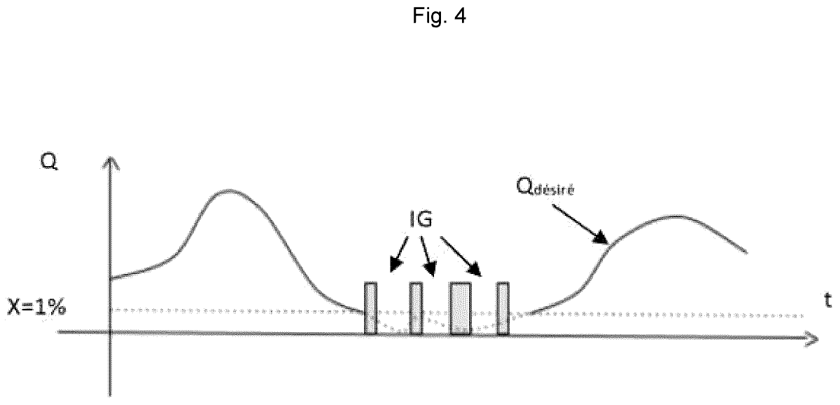

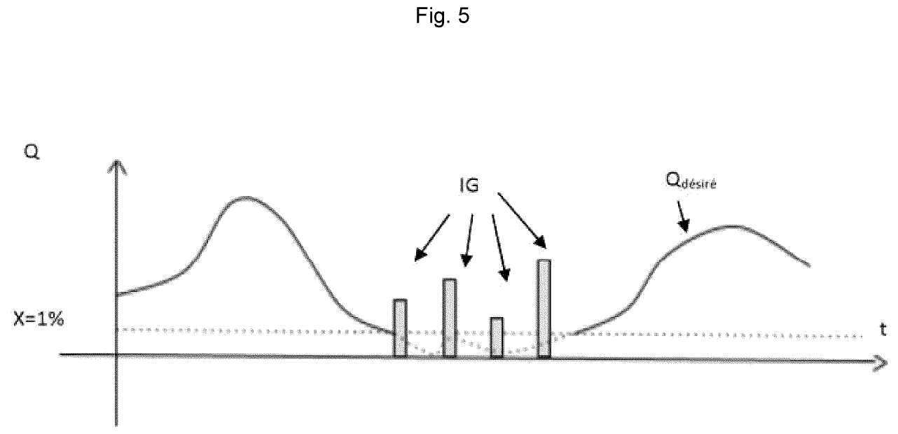

This is shown in FIG. 4 and FIG. 5, which show the gas pulses IG or bursts delivered by the proportional solenoid valve 42 in such a way as to achieve the desired gas flow rate (Q_{desired}) downstream of the proportional solenoid valve 42.

In these FIG. 4 and FIG. 5, the first operating range, which is non-linear, ranges from 0 to 1% (x %=1%) and the second operating range, which is linear, ranges from 1 to 90%, approximately.

It can be seen in FIG. 4 that it is possible to fix, adjust or modulate the flow rate of supplied gas by adjusting the width of each gas pulse IG (i.e., the width of the IG's on the graph), which corresponds to the time period (i.e. duration) during which the proportional solenoid valve 42 is open. Indeed, the longer the time period during which the proportional solenoid valve 42 is open, the higher the quantity of gas (i.e., flow rate) that can pass through it during each pulse IG, and vice versa.

Moreover, as can be seen in FIG. 5, it is also possible to fix, adjust or modulate the flow rate of supplied gas by adjusting the amplitude of each gas pulse IG (i.e., the height of the IG's on the graph), which corresponds to the degree of opening of the proportional solenoid valve 42. Indeed, the larger the degree of opening (x %) of the proportional solenoid valve 42 (for example being close to 1% when x=1%), the larger its opening/passage cross section and, therefore, the more gas can pass through it during each pulse IG, and vice versa.

By varying the amplitudes and/or the widths of the gas pulses, a quantity of NO that corresponds to the desired gas flow rate (Q_{desired}) can be delivered.

This control of the amplitude or duration of the gas pulses IG is carried out by the operating means 50 which operate the proportional solenoid valve 42, in particular depending on the desired gas flow rate, which depends on the flow rate of the flow of gas containing oxygen (>approximately 20% vol.) delivered by the ventilator 30, measured by the flow rate sensor 24, such as a flow of air or of O₂/N₂ gaseous mixture, the concentration of NO in the NO/N₂ gaseous mixture supplying the apparatus 1 and the desired dosage, i.e., the desired NO content in the final gaseous mixture that results from mixing the flow of gas containing oxygen (>approximately 20% vol.) delivered by the ventilator 30 and the flow of NO/N₂ from the apparatus 1.

The apparatus for supplying NO 1 and/or the installation for supplying gas 10 of the invention are used to administer, by inhalation, in particular via a tracheal intubation tube, a gaseous mixture comprising between 1 and 80 ppmv of NO, typically of the order of 10 to 20 ppmv of NO, and at least approximately 20% vol. of oxygen, to a patient in need of it (e.g., an adult, child, adolescent or newborn, including a premature baby), typically a patient (i.e., a human being) suffering from pulmonary hypertension and/or hypoxia that may cause pulmonary vasoconstriction or the like, for example caused by pulmonary diseases or disorders such as PPHN (persistent pulmonary hypertension of the newborn) or ARDS (acute respiratory distress syndrome), or those caused by heart surgery in which the patient is put on extracorporeal circulation (ECC).

What is claimed is:

1. Apparatus for supplying NO (1), comprising:
a main gas circuit (40; 40.1, 40.2) for conveying an NO/N₂ gaseous mixture,
flow rate control means (42, 43) comprising at least one main solenoid valve (42), arranged on the main gas circuit (40; 40.1, 40.2), and
operating means (50) configured to control at least said at least one main solenoid valve (42) in order to control the flow rate of NO/N₂ gaseous mixture passing through said at least one main solenoid valve (42),
characterized in that:
the main solenoid valve (42) is a proportional solenoid valve having degrees of opening of between 0 and 100%, and is configured to function in at least two given operating ranges comprising at least:
a first operating range corresponding to degrees of opening of the main solenoid valve (42) of between 0 and x % where: 0<x %≤10%, and
a second operating range corresponding to degrees of opening of the main solenoid valve (42)>x %, and
the operating means (50) are configured to control said main solenoid valve (42) in pulsed mode in the first operating range so as to deliver successive gas pulses (IG).

2. Apparatus according to claim 1, characterized in that the operating means (50) are configured to control said main solenoid valve (42) in proportional mode in the second operating range.

3. Apparatus according to claim 1, characterized in that the first operating range corresponds to degrees of opening of the main solenoid valve (42) of between 0 and x % where: 0<x %≤5%.

4. Apparatus according to claim 1, characterized in that the operating means (50) are configured to fix or adjust a duration or amplitude of each gas pulse (IG) delivered in pulsed mode, in order to control the flow rate of gas passing through said main solenoid valve (42).

5. Apparatus according to claim 4, characterized in that the operating means (50) are configured to adjust the pulse duration (IG) of each pulse by controlling the duration of opening of the proportional solenoid valve (42).

6. Apparatus according to claim 4, characterized in that the operating means (50) are configured to adjust the pulse duration (IG) of each pulse by controlling the amplitude of each pulse (IG) by controlling the degree of opening (x %) of the proportional solenoid valve (42).

7. Apparatus according to claim 5, characterized in that the duration of opening of the proportional solenoid valve (42) is between 5 and 200 msec.

8. Apparatus according to claim 5, characterized in that, in pulsed mode, the operating means (50) are configured to control the proportional solenoid valve (42) to deliver successive gas pulses in which each gas pulse has a non-zero pulse duration (di) during which the proportional solenoid valve (42) is in a partially open position with a degree of opening x %, such that x %<10%, so as to allow a given quantity of NO/N₂ mixture to pass through during said pulse duration (di).

9. Apparatus according to claim 8, characterized in that the pulse time period (di) of each gas pulse (IG) is of non-zero variable duration.

10. Apparatus according to claim 9, characterized in that the pulse time period (di) of each gas pulse (IG) is between 5 and 200 msec.

11. Apparatus according to claim 1, characterized in that it further comprises a bypass circuit (41) which is fluidically connected to the main gas circuit (40; 40.1, 40.2), upstream and downstream of the flow rate control means (42, 43) and comprises secondary flow rate control means (45, 46) comprising an all or nothing secondary solenoid valve (45) and a fixed flow rate device (46).

12. Apparatus according to claim 11, characterized in that the all or nothing secondary solenoid valve (45) is controlled in pulsed mode by the operating means (50).

13. Apparatus according to claim 12, characterized in that the secondary solenoid valve (45) is configured to adopt only two operating positions comprising an open position allowing the flow of NO/N₂ mixture to pass through and a closed position preventing any flow of NO/N₂ mixture from passing through.

14. Apparatus according to claim 11, characterized in that the fixed flow rate device (46) comprises a calibrated orifice device delivering a predetermined fixed flow rate (Q_{fixed}) of between 0.1 and 2 L/min.

15. Installation for supplying NO (10) to a patient, comprising:
at least one source (11) of NO/N₂ mixture,
an apparatus for supplying NO (1) according to claim 1, fluidically connected to said at least one source (11) of NO/N₂ mixture,
a medical ventilator (30) configured to supply a respiratory gas containing oxygen, typically at least approximately 20% oxygen, such as air or an N₂/O₂ mixture,
a patient circuit (20) to which the apparatus for supplying NO (1) and the medical ventilator (30) for supplying said patient circuit (20) with said NO/N₂ gaseous mixture and said respiratory gas containing oxygen, respectively, are fluidically connected, and
a flow rate sensor (24) arranged on the patient circuit (20) and configured to determine and supply to the operating means (50) of the NO delivery device (1) at least one measurement signal representative of the gas flow rate within the patient circuit (20).
